(19) United States
(12) Patent Application Publication
(10) Pub. No.: US 2025/0104849 A1
(43) Pub. Date: Mar. 27, 2025

(54) AUTOMATED, SELF-SERVICE CLINICAL TRIAGING AND MEDICAL APPOINTMENT SERVICE TYPE SELECTION

(71) Applicant: AndorHealth, LLC, Orlando, FL (US)

(72) Inventors: Raj Toleti, Orlando, FL (US); Balavignesh Thirumalainambi, Orlando, FL (US)

(73) Assignee: AndorHealth, LLC, Orlando, FL (US)

(21) Appl. No.: 18/726,202

(22) PCT Filed: Jan. 9, 2023
(86) PCT No.: PCT/US2023/010440

§ 371 Date: Jul. 2, 2024

Related U.S. Application Data
(60) Provisional application No. 63/297,479, filed on Jan. 7, 2022.

(51) Int. Cl. G16H 40/20; G16H 10/60; G16H 50/30; G16H 80/00

(52) U.S. Cl. CPC G16H 40/20; G16H 10/60; G16H 50/30; G16H 80/00

(57) ABSTRACT

An automated triage system is provided having at least one memory configured to store programmable instructions and at least one processing device in communication with the at least one memory, wherein the at least one processing device, upon execution of the programmable instructions is caused to automatically receive demographics data for a patient; access or receive a medical history for the patient; receive a medical complaint about a present medical condition; triage the patient based on the demographic data, the medical history, and the medical complaint to determine a level of urgency of treatment needed by the patient; select an appointment service type for servicing the patient based on results of the triage to reduce unnecessary emergency room visits; provide a response to the patient including the appointment service type selected; and initiate an appointment of the appointment service type selected for the patient that is available to the patient.

### CROSS REFERENCE TO RELATED APPLICATIONS

This is a national phase application under 35 U.S. C. § 371 of PCT patent application PCT/US2023/010440, filed on Jan. 9, 2023, which claims priority to U.S. Provisional Patent Application Ser. No. 63/297,479, filed Jan. 7, 2022, each of which is incorporated herein by reference in its entirety.

### FIELD

This disclosure relates to health information technologies, and more particularly to methods and systems for automatic clinical triaging and selection of appointment service types for receiving medical care.

### BACKGROUND

Lack of intervention to guide the patients to appropriate care settings is one of the key issues facing the US, including several other developing countries. A study conducted in 2017 revealed that more than half of the care provided in the U.S. is through emergency visits, where they could have been triaged to other lower acute care settings. This practice increases the cost of providing care and utilizes valuable resources. This is neither sustainable nor effective.

Lack of triage to appropriate care has been an issue throughout the U.S. More than $32 billion is wasted every year due to inappropriate use of the Emergency Room. It was identified that ⅔rds of the 27 million visits to the ER are avoidable. Many Americans depend on the emergency room and since an average ER visit can cost more than $2,000, it is an issue that needs to be solved for providing appropriate and better care for all Americans, especially the underserved community and for the benefit of the economic health of the country. It is notable that this issue has only been exacerbated with the COVID-19 global pandemic.

In a current workflow, when a patient shows up in the ER, there is little or no intervention to check if the ER visit is warranted. Depending on the severity of the patient's condition, the patient may wait for more than an hour and in some cases even multiple hours before they move to the registration step. The average waiting time in the ER is about 40 minutes. Typically, after this waiting period, the patient's intake information, such as demographics, insurance, chief complaint, and other relevant details would be obtained by the registration staff at the ER. A triage nurse would then evaluate the patient and would collect vitals such as temperature, blood pressure and other clinically relevant information from the patient. The patient is then triaged to the examination room, where the ER physician will evaluate the patient and will decide the course of action.

Such conventional methods and systems have generally been considered satisfactory for their intended purpose. However, there is still a need in the art for improved triaging systems. The present disclosure provides a solution for this need.

### SUMMARY

The purpose and advantages of the below described illustrated embodiments will be set forth in and apparent from the description that follows. Additional advantages of the illustrated embodiments will be realized and attained by the devices, systems and methods particularly pointed out in the written description and claims hereof, as well as from the appended drawings. To achieve these and other advantages and in accordance with the purpose of the illustrated embodiments, in one aspect, disclosed is an automated triage system having at least one memory configured to store a plurality of programmable instructions and at least one processing device in communication with the at least one memory. The at least one processing device, upon execution of the plurality of programmable instructions is caused to perform a disclosed method. The method includes automatically receiving demographics data for a patient, accessing or receiving a medical history for the patient, and receiving a medical complaint about a present medical condition. The method further includes triaging the patient based on the demographic data, the medical history, and the medical complaint to determine a level of urgency of treatment needed by the patient. The method further includes selecting an appointment service type for servicing the patient based on results of the triage to reduce unnecessary emergency room visits, providing a response to the patient including the appointment service type selected, and initiating an appointment of the appointment service type selected for the patient that is available to the patient.

In one or more embodiments, the appointment service type can include a primary care physician type, a specialist type, an emergency virtual visit type, or an emergency room type.

In one or more embodiments, the at least one processing device can be further caused to prompt the patient, based on at least one of the demographic data and the medical record, for key clinical data, wherein receiving the medical complaint can include receiving the key clinical data.

In one or more embodiments, the key clinical data can include a primary medical complaint and/or a secondary medical complaint.

In one or more embodiments, the at least one processing device can be further configured to generate a graphical user interface (GUI) configured to be displayed on a display device for interacting with a user and receiving user input.

In one or more embodiments, the at least one processing device can be further caused to determine if the patient is an existing patient or a new patient, wherein prompting the patient can be further based on the determination.

In one or more embodiments, the at least one processing device can be further caused to provide a full record of care for analysis, to the patient, and/or to a provider of the patient, upon determining that the patient is an existing patient.

In one or more embodiments, the at least one processing device can be further caused to compare the user inputs to a triage database, wherein selection of the appointment service type can be a function of a result of the comparison.

In one or more embodiments, the at least one processing device can be configured to apply machine learning (ML) to select the appointment service type.

In one or more embodiments, the at least one processing device can be configured to automatically recommend a service provider from a plurality of candidate service providers as a function of one or more of patient affiliation with a service provider of the plurality of candidate service providers; acceptance of patient insurance, location of the service provider; availability of one or more of a specialized practitioner, facilities, and/or equipment; cost quote or estimate; type of payment accepted; and patient preference.

In one or more embodiments, initiating the appointment can include outputting at least one control signal to order, allocate, or reserve at least one resource for performance of the appointment.

In one or more embodiments, initiating the appointment can include prioritizing patients based on results of the triage and tracking wait time of the patient at the appointment as a function of prioritization of the patient.

In one or more embodiments, initiating the appointment can include prioritizing patients based on results of the triage and outputting at least one control signal to order, allocate, or reserve at least one resource for performance of the appointment as function of the prioritization of the patients.

In one or more embodiments, the appointment is a virtual visit and initiating the appointment includes automatically providing a link to patient selected invitees to join the virtual visit.

In accordance with an additional aspect of the disclosure, the disclosed method is provided.

In accordance with a further aspect of the disclosure, a disclosed method includes displaying a patient identification graphical user interface (GUI) having one or more fields configured to receive identification and/or location information from a patient, displaying a triage GUI having one or more fields configured to receive medical complaint information; determining a virtual care condition existence based on the medical complaint information; displaying a virtual visit screen where a virtual care condition exists; and displaying an alternate screen where no virtual care condition exists.

In one or more embodiments, the alternate screen can include an emergency department screen configured to provide instruction to the user to go to an emergency department and/or emergency department information based on location information if an emergency condition exists.

In one or more embodiments, the alternate screen can include a primary care physician screen instructing a user to contact their primary care physician if no emergency condition or virtual care condition exists.

In accordance with a further aspect of the disclosure, at least one non-transitory computer-readable medium storing executable code is provided. The executable code is configured to cause at least one processor to perform each of the disclosed methods. In accordance with another aspect of the disclosure, a system is provided. The system includes one or more computer modules configured to display a patient identification GUI having one or more fields configured to receive identification and/or location information from a patient, display a triage GUI having one or more fields configured to receive medical complaint information, determine a condition type based on the medical complaint information, wherein the condition type is one of a virtual care condition, an emergency condition, or a primary care condition, display a virtual visit screen if a virtual care condition exists, display an emergency department screen if an emergency condition exists, and display a primary care physician screen if a primary care condition exists.

In one or more embodiments, the virtual care screen can include a join waiting room button configured to place the user into a virtual waiting room for a virtual care visit.

### BRIEF DESCRIPTION OF THE DRAWINGS

A more detailed description of the disclosure, briefly summarized above, may be had by reference to various embodiments, some of which are illustrated in the appended drawings.

While the appended drawings illustrate select embodiments of this disclosure, these drawings are not to be considered limiting of its scope, for the disclosure may admit to other equally effective embodiments.

FIG. 1A shows a flow diagram illustrating an example portion of a method performed by an automated triage system, in accordance with embodiments of the disclosure.

FIG. 1B shows an example screenshot of a graphical user interface (GUI) provided by the automated triage system and displayed to a user for beginning a triage process, in accordance with embodiments of the disclosure.

FIG. 2 shows an example screenshot of a patient demographic information GUI provided by the automated triage system and displayed to a user for viewing and/or entering demographic information, in accordance with embodiments of the disclosure.

FIG. 3 shows an example screenshot of the GUI provided by the automated triage system to prompt the user with one or more triage questions, in accordance with embodiments of the disclosure.

FIGS. 4-6 show example screenshots of the GUI provided by the automated triage system and displayed to a user with a triage results directing the patient to an appropriate level of care, in accordance with embodiments of the disclosure.

FIG. 7 shows an example screenshot of the GUI provided by the automated triage system to a provider or staff of a virtual waiting room, in accordance with embodiments of the disclosure.

FIG. 8 shows an example screenshot of the GUI provided by the automated triage system for accessing a registration page for a user to complete registration in preparation of an emergency virtual visit, in accordance with embodiments of the disclosure.

FIG. 9 shows an example screenshot of the GUI provided by the automated triage system providing a page for the user to update demographic data for the patient, in accordance with embodiments of the disclosure.

FIG. 10 shows an example screenshot of the GUI provided by the automated triage system to gather additional information from a user about the patient's current symptoms and other data related to the patient's medical complaint, in accordance with embodiments of the disclosure.

FIGS. 11-13 shows example screenshots of the GUI provided by the automated triage system having a dashboard included in a view for care providers and staff of the virtual waiting room for viewing patient data and triage responses, in accordance with embodiments of the disclosure.

FIG. 14 shows a portion of an example triage database storing triage data is shown, in accordance with embodiments of the disclosure.

FIGS. 15, 15A, and 15B show a flow diagram of an example method performed by the automated triage system, in accordance with embodiments of the disclosure.

FIG. 16 shows example screenshots provided to a patient and a provider during a virtual visit.

FIG. 17 is a block diagram of the automated triage system having an exemplary computer system, in accordance with embodiments of the disclosure.

Identical reference numerals have been used, where possible, to designate identical elements that are common to the figures. However, elements disclosed in one embodiment may be beneficially utilized on other embodiments without specific recitation.

### DETAILED DESCRIPTION

Reference will now be made to the drawings wherein like reference numerals identify similar structural features or aspects of the subject disclosure. For purposes of explanation and illustration, and not limitation, an illustrative view of an embodiment of a system and method in accordance with the disclosure is shown in the various Figures.

Embodiments relate to the healthcare field, more specifically to a health information technology solution for improvement in processing patients seeking immediate care. In accordance with certain embodiments, automated clinical triaging can be used to effectively triage patients to appropriate care. This technology can aid in automatically collecting key data points from the patients, automatically triaging the patients based on the collected data points, and automatically directing them to a virtual or in-person option based on their specific needs. The patient is only directed to an emergency room for medical care when appropriate. An appointment for medical care of the appointment service type (e.g., primary care service, specialist service, virtual emergency room service, or physical emergency room service) to which the patient is directed can be automatically initiated.

Automatic triage of a patient includes an automated assessment of the patient in order to determine a level of urgency for receiving treatment and a nature of the treatment needed. The appointment service type is selected based on the determined level of urgency.

In one or more embodiments, the appointment service type can further be selected based on demographics and/or location of the patient, as well as availability and/or acceptance of certain insurances by facilities and/or providers that provide the treatment needed.

The automated triage service can be a centralized service that triages eligible patients that may be affiliated and/or located near a variety of multiple healthcare service providers of ERs, emergency virtual visits, PCPs and specialists. The automated triage service can automatically recommend ER, emergency virtual visits, PCPs and specialists based on patient affiliation with these service providers, acceptance of patient insurance, location of the service provider, availability (e.g., of specialized personnel, facilities, and/or equipment), cost quote or estimate, type of payment accepted, patient preference, etc. Once the recommendation to particular service provider is made, the automated triage service can output a control signal to order, allocate, or reserve resources (medications, personnel, room, equipment, etc.).

Embodiments can eliminate the need for the registration staff, nurse, or even the provider from seeing a patient who might have presented to the ER for non-emergent issue. The digital triage solution can be presented to the patient as soon as the patient shows up to the ER or an ER triaging facility or before they arrive at the ER or the ER triaging facility. The patient or a caretaker can immediately complete their intake steps presented to them through any digital device. Embodiments can provide an intake process that can include asking a list of key questions (e.g., presented on a GUI) that can identify if the patient is already subscribed to the automatic triage system or registered with an associated healthcare system that already stores information about the patient. If patient data (including demographic and/or medical history information about the patient) is already stored and accessible to the automated triage system it can be used to populate any necessary information. The patient can be prompted to enter any information that is not populated.

Furthermore, the relevant clinical information (e.g., patient data and triage responses) that is stored can also be surfaced in the virtual session to be available to the provider via the software that provides the virtual session; allowing the provider to work in fewer software applications. In this way, a provider device used by the provider for participating in the emergency virtual session can have fewer applications opened. The provider can consult fewer applications to access relevant information. This provides technical advantages, by reducing the processing load of the provider's device and reducing the amount of data that needs to be displayed on a small display screen. The technical advantages further include easier access to information by the provider without a need to access information from multiple applications. Instead, the provider can access only the application used for the providing the virtual visit, without the need to move to another application to access information. This can save time, which is important for providing the needed immediate care to the patient and reducing wait time for other patients waiting for virtual appointments.

The system can also triage by asking a set of key questions about the patient's medical complaint (e.g., symptoms and other issues). The system can further ask a set of key questions to fill in any information still needed about medical history. The triage responses to the key questions can include key clinical data.

Embodiments can include a complete automated triaging solution that can use artificial intelligence (AI), including logic and/or machine learning (ML), to select and guide patients to appropriate lines of care. In one or more embodiments, the automated triaging solution can be accessed by patients well before they show up at the emergency room. Embodiments can also validate the need for the visit and help them to move to the appropriate next point of care. A workflow utilized by an automated triage system by certain embodiments can include:
(1) Identifying the patient and their record of care that is available to the automated triage system.
(2) Identifying the severity of the patient's condition.
(3) Triaging the patient to determine urgency of treatment and select an appropriate care setting and/or to direct the patient out of an inefficient care scenario.

Embodiments can utilize any suitable hardware and/or software module(s) configured to perform the disclosed function(s) herein, e.g., to display one or more graphical user interfaces (GUI) as shown and/or described herein (e.g., on a patient-side device or caregiver-side device or both).

### Collecting Patient Data

A starting point of the automated triaging process can begin on any suitable digital location (a website, an installed application (app), or pushed/pulled via Text/Email to/from patients). The patient can be presented with a patient identification GUI having an initial set of demographic fields (e.g., name, date of birth, sex, patient identification (ID) number (optional, e.g., can be a national or proprietary ID), phone, residential address, email, present location). Information provided in these fields is used to match the patient to an existing patient record in a patient record system. Using this matching process, the patient record system can access the patient's record, such as to access all or a portion of the patient's medical history stored in the patient's patient record. Once the patient's record is surfaced and accessible, it can be available to software of the automated triage system for analysis and display to a user (the patient, the patient's caretaker, and/or a provider or staff handling appointments), without the need for the software or users to display, view, or access data from software applications external to the automated triage system, such as the patient record system.

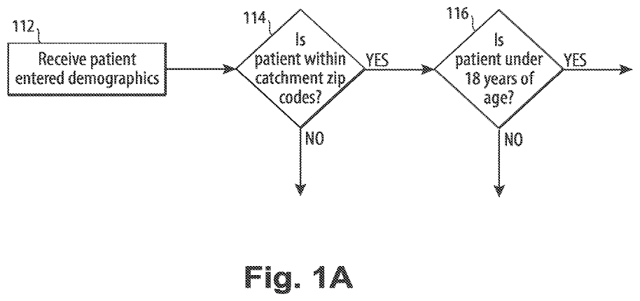

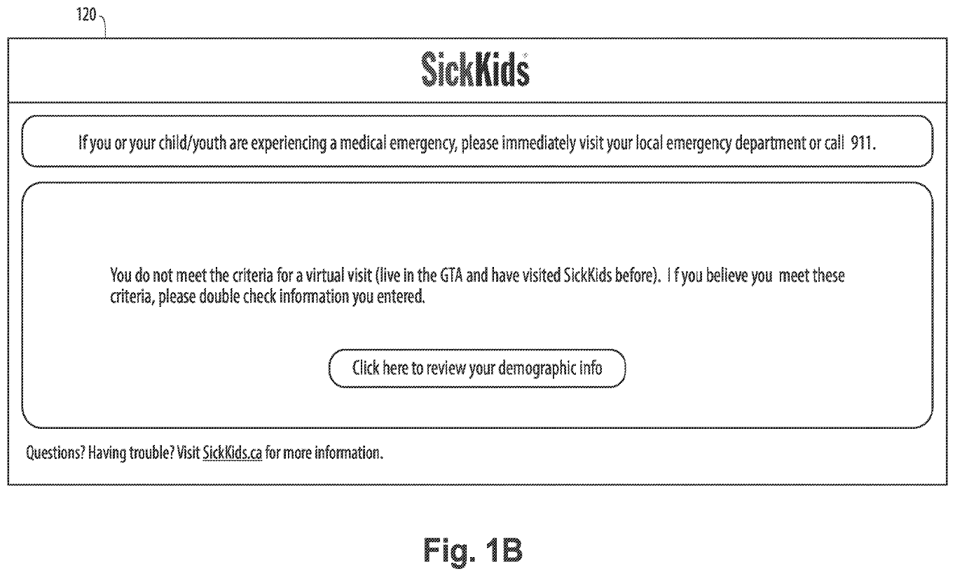

In certain embodiments, e.g., as shown in FIGS. 1A and 1B, there can be an age and/or zip code review at a suitable portion in the workflow, e.g., after receiving the patent's demographics data and before collecting medical data or evaluating urgency of the patient's medical condition, without limitation to a particular location in the workflow. The workflow of the automated triage system can depend on whether the patient resides within and/or is currently located within a zip code area that uses the automated triage system, in which after triaging the patient, referral for the patient to emergency services may be limited to a list of emergency departments (EDs) where the patient is located. A determination can also be made at this point whether the patient is subscribed to the service provided by the automated triage system. Regardless of the patient's location or residence, when virtual emergency services are recommended, if demographics reveal that the patient is subscribed to the automated triage system, the automated triage system can recommend and/or initiate a virtual emergency visit.

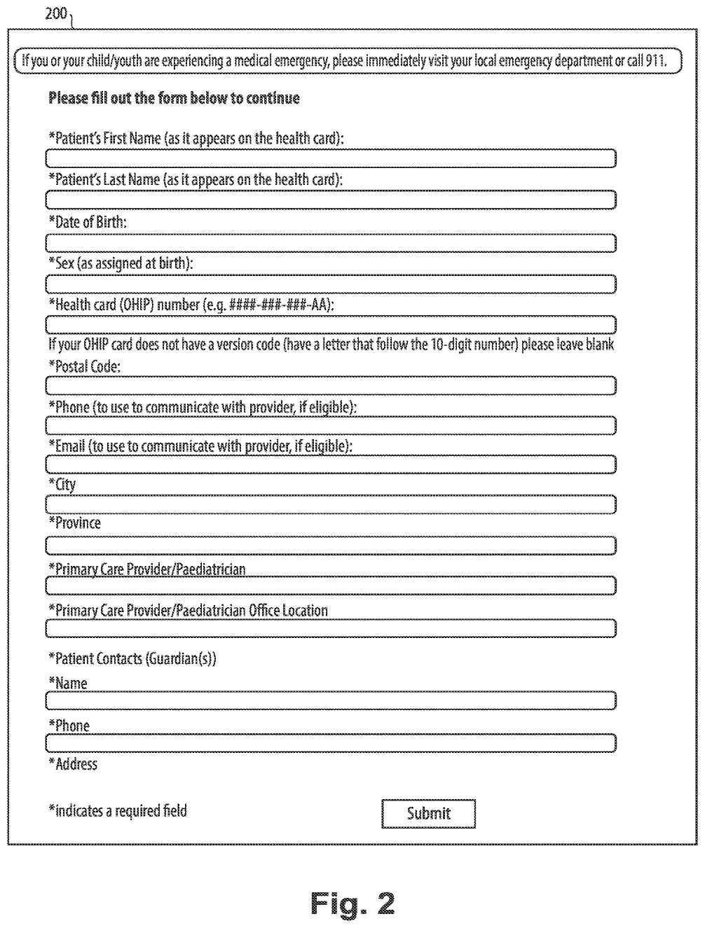

Matching the patient to an existing patient record can include accessing externally stored electronic medical records (EMRs) or internally stored records, such as by utilizing an application programing interface (API). The API can be used to automatically populate demographic and/or medical history information about the patient, such as by interfacing with an EMR system storing the patient's EMR using industry standard interfacing standards and presenting them in an appropriate format and layout for use by the automated triage system, e.g., as depicted in FIG. 2.

If the patient is new patient or demographic or medical information for the patient are not stored by or accessible to the automated triage system, the automated triage system can then automatically prompt the patient or the patient's caretaker to provide information that is still needed, such as the following:
1. Address
2. Healthcare Provider Information
3. Guardian Information

The automated triage system, if appropriate, can use these additional data points to create a record for the patient in a healthcare system that will be providing the medical care to the patient as directed by the automated triage system. The new patient record can be created using an API, e.g., a proprietary APis, a Fast Healthcare Interoperability Resources (FHIR) based API, or Health Level 7 (HL7).

### Collecting Triage Data

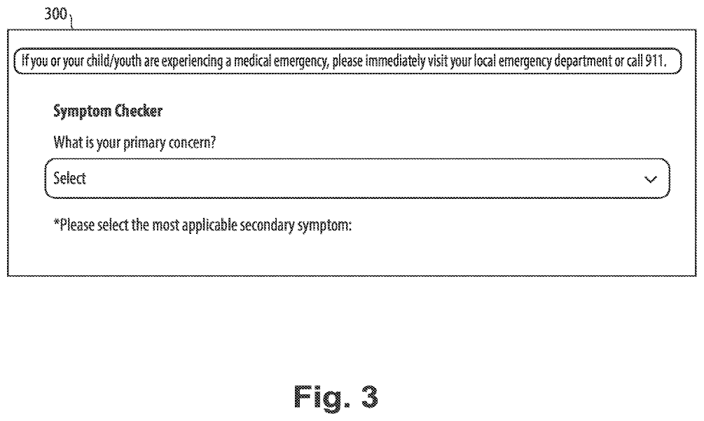

After this step, a user (the patient or a caretaker of the patient) can be prompted (e.g., via a triage GUI or an audio prompt) to answer triage questions by entering at least one medical complaint A primary medical complaint can answer the following example triage question, as shown in FIG. 3:

### What is Your Primary Concern?

The triage question can include a prompt to enter the medical complaint. The prompt can include a dropdown menu with selections for a primary medical complaint (e.g., animal/human bite, asthma attack, back pain, etc.). Alternatively and/or additionally, the prompt to enter the primary medical complaint can include a user entry field for a text input that uses natural language.

Based on the primary medical complaint entered by the user, the automated triage system can ask further triage questions, e.g., via a prompt (e.g., via the GUI or audio prompts) to enter a secondary medical complaint. The prompt to enter the secondary medical complaint (depicted in FIG. 3) can be used to further validate whether a patient is optimal for in-person or virtual care. Alternatively and/or additionally to selected dropdown menus, the prompt to enter the secondary medical complaint can include a user entry field for a text input that uses natural language.

### Triage—Identifying Level of Urgency [o]f the Patient's Condition

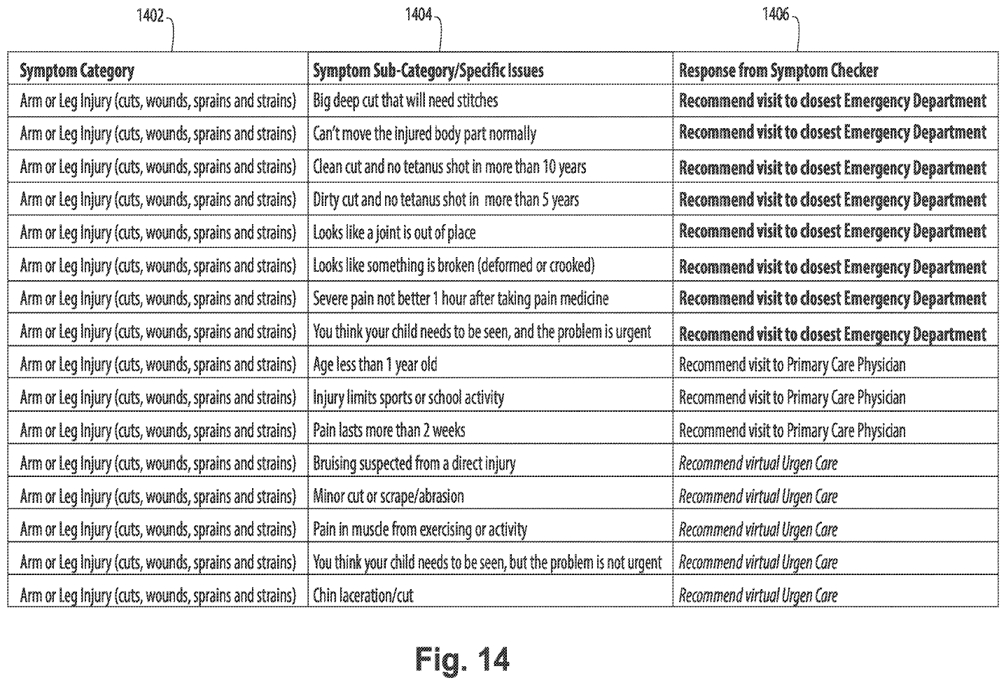

The responses to the triage questions are processed by an algorithm and/or machine learning (ML) to triage the patient by determining a level of urgency of the patient's condition in order to determine if an emergency room visit is warranted. The algorithm and/or ML applied can include a comparison of the responses to the triage questions to data stored in a triage database. An example of a portion of the triage database with logic employed by the algorithm is shown in FIG. 14. Triage responses are compared to triage data stored in the triage data base related to medical complaints, and an associated recommendation is selected.

With reference to FIG. 14, a portion of triage data stored in the triage database that was selected based on a patient's triage responses is shown. The triage data is organized into a category column 11701 having category entries, a sub-category column 11702 having sub-category entries, and a response column 11703 having response entries are shown. Category column 11701 lists a symptom category selected from a plurality of candidate symptom categories, e.g., by the algorithm, based on responses to the dropdown menus for entering the primary medical complaint. The symptom category can also be selected based on responses to the dropdown menus for entering the secondary medical complaint and/or patient data, including demographic and/or medical history for the patient.

Sub-category column 11702 lists different possible sub-category entries for a symptom category specified. Each sub-category entry describes a symptom sub-category and/or a specific issue. During a triage process, after a category is selected, a best-fit sub-category is selected using the secondary medical complaints are. The sub-category entry can also be selected using the patient data.

Each sub-category entry has an associated response entry that specifies an in-person and/or virtual appointment service type to recommend to the patient for the category and sub-category entries selected. Three different appointment service types are shown, without limitation to only these particular service types, namely appointments for service by an emergency department, a PCP, or virtual urgent care.

FIG. 14 shows a triage process in which the category (arm or leg injury (cuts, wounds, sprains and strains) was selected based at least on responses to the triage question(s) presented and possibly based on patient data as well. Once a sub-category entry is selected, e.g., based on the triage questions and/or patient data, the recommendation provided in the associated response entry is displayed (e.g., via the GUI) or otherwise communicated to the user.

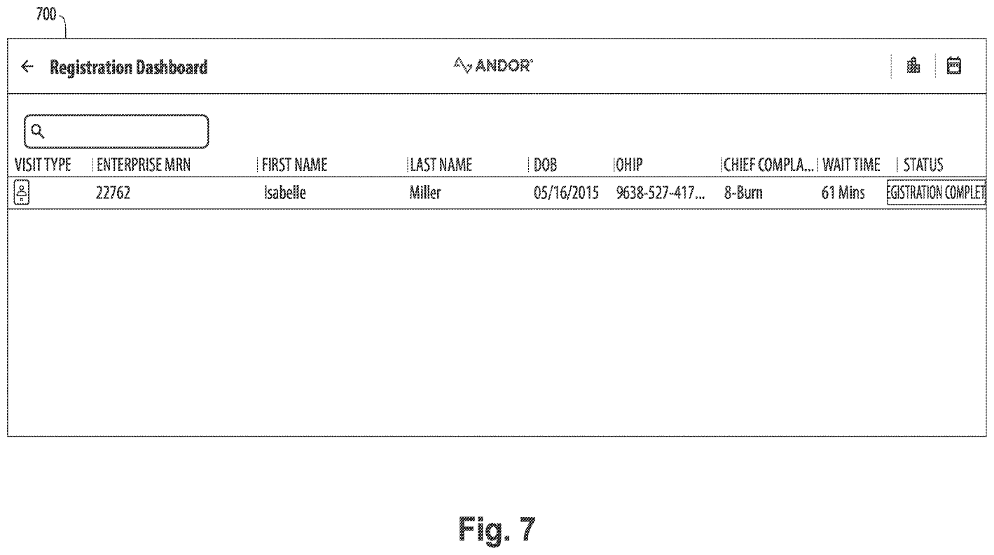

In certain embodiments, the automated triage system can display a care provider side GUI. For example, the care team can be presented with a dashboard that shows the real-time interaction of patients with the automated triage system and shows the care team at what stage the patients are at in the process. The dashboard can show the care team if the patient is either stuck at a step or if they have completed the steps and are now waiting for a care team member to start examining them. An example of a provider side GUI is shown in FIG. 7, for example.

### Directing the Patient to an Appropriate Care Setting

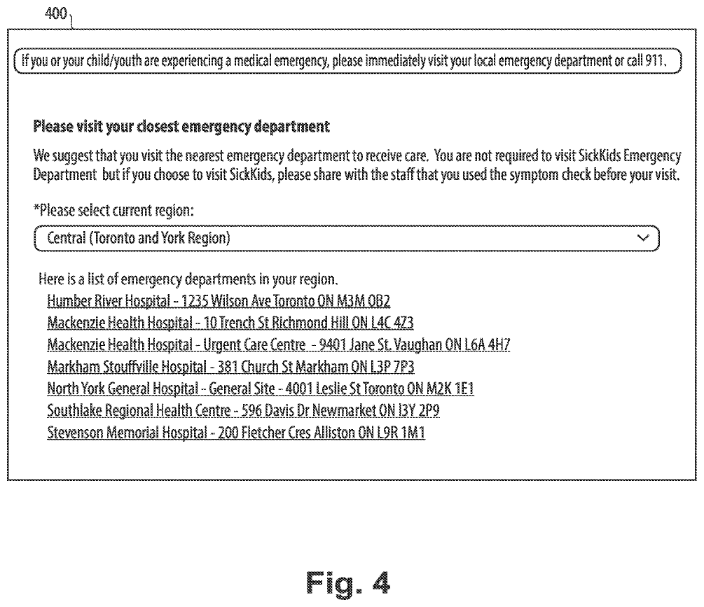

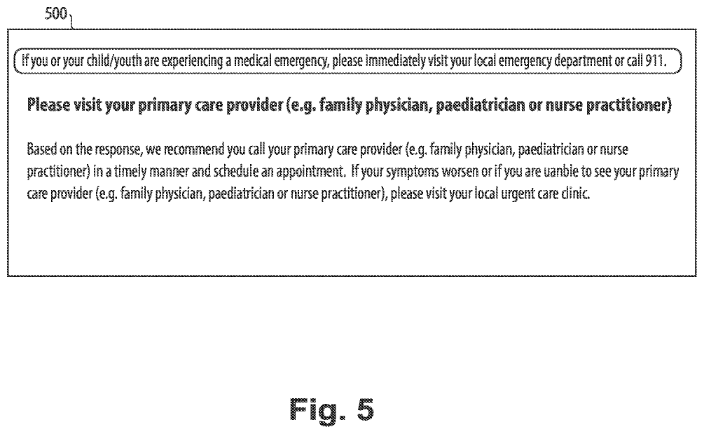

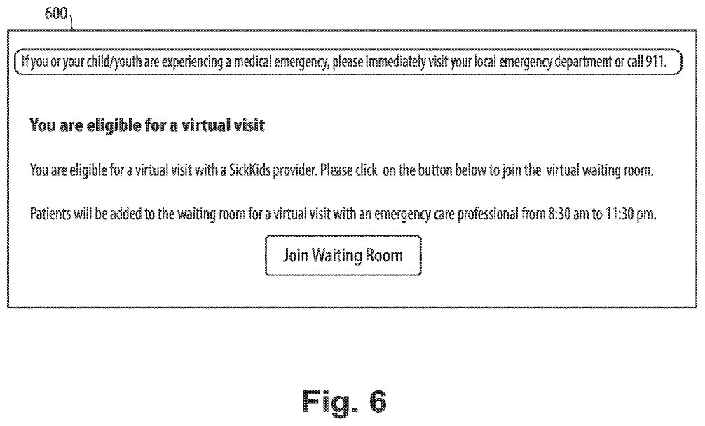

Once the algorithm has recommended an appointment service type to the patient based on responses to the triage questions and the patient data, the system can guide the patient to:
a. go to an emergency department in person that is close-by (as depicted in FIG. 4);
b. visit a PCP (as depicted in FIG. 5); or
c. engage in a virtual visit with the emergency department (as depicted in FIG. 6) or other suitable provider for urgent care services (also referred to as an emergency virtual visit).

### Actions Triggered by Recommendation

If the automated triage system identifies that the patient should be admitted to the ER, the automated triage system can immediately alert a triage nurse through a text, an email, or other suitable communication channels and reserve physical resources (e.g., personnel, space and/or equipment) as needed to move the patient to collect vitals and move the patient to an examination or treatment room. The automated triage system can prioritize a patient based on his triage responses and can further track the patient's waiting time in view of the prioritization. Additionally or alternatively, the automated triage system can allocate resources for an appointment of the patient as a function of the patient's priority.

If the automated triage system determines that the ER visit is not necessary, the automated triage system can immediately present the patient with a list of appointment service types, such as for primary care, specialist care, or an emergency virtual visit, along with a list of provider availability. The care team's dashboard disclosed above can indicate to the care team that the patient has been triaged to another care setting, for example. The patient's GUI can allow the patient to select invitees to join the primary emergency virtual visit and automatically invite the invitees to join the virtual visit by using a link provided to their phone or email.

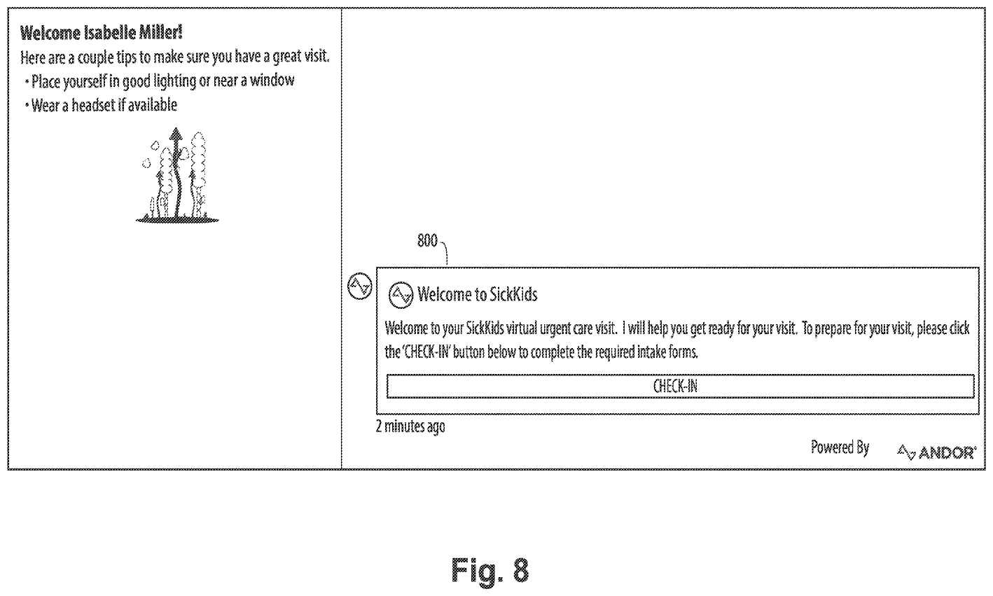

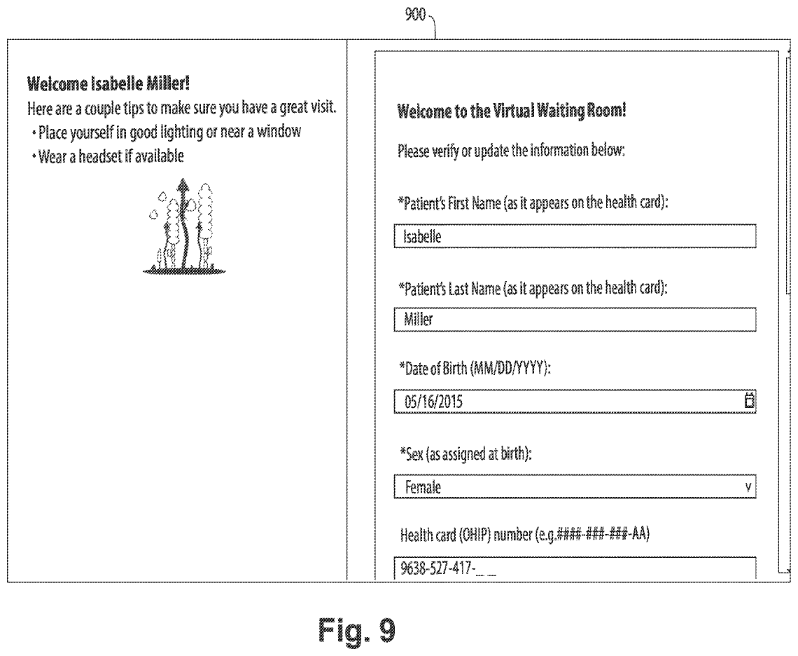

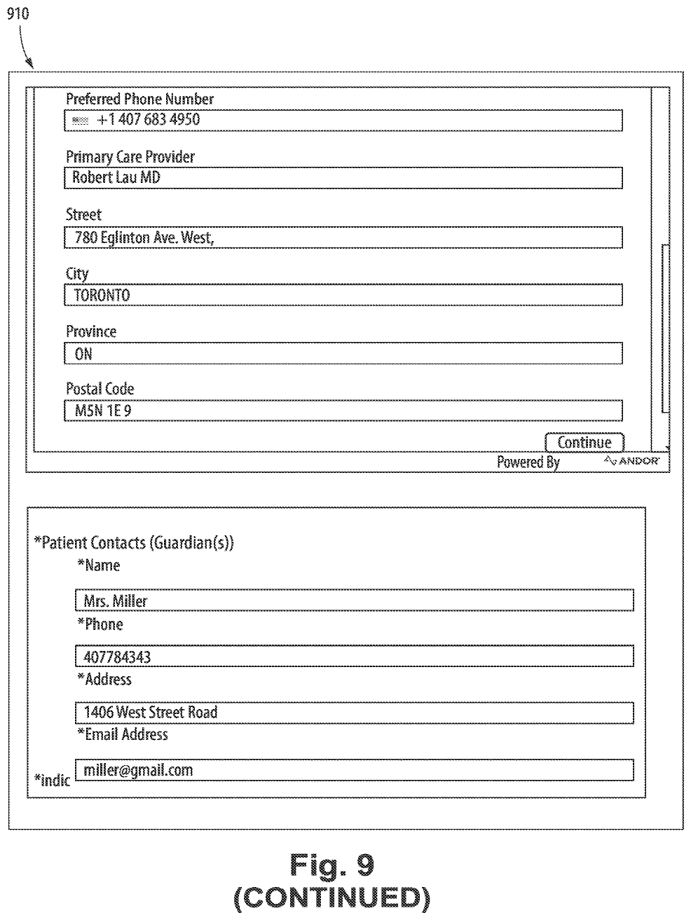

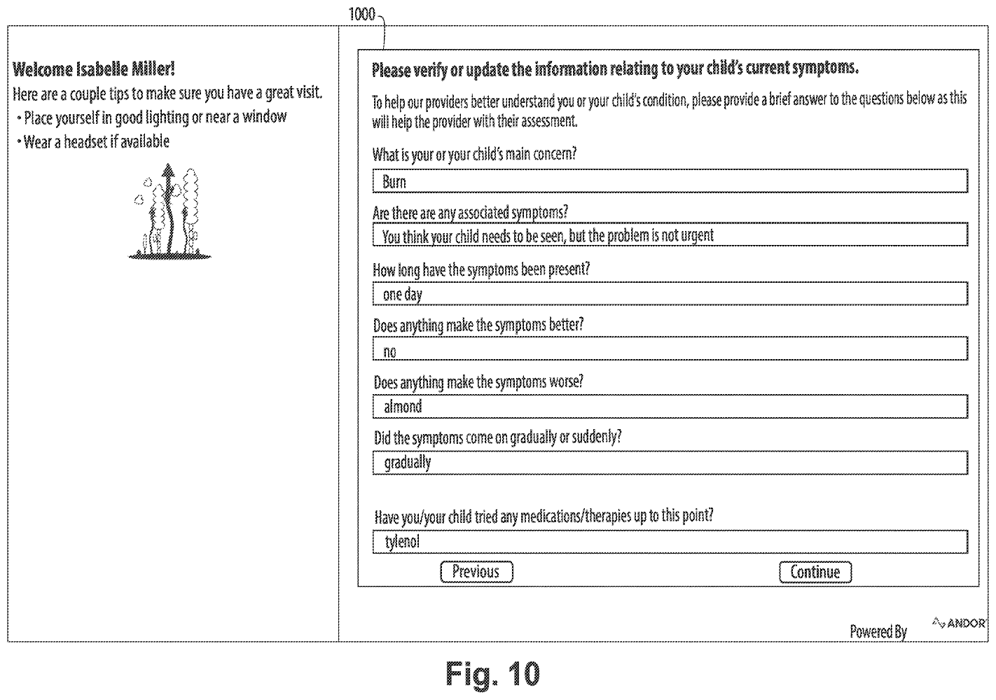

The automated triage system allows the patient to follow the recommended avenue of care and continue to set up the appropriate follow up care without having to wait for an ER physician. The automated triage system can also set up a virtual emergency care visit where the patient is presented with a self-service option to schedule an appointment with the next provider of care. The patient can be taken to a waiting room (e.g., as shown in FIG. 8) in order to proceed through a workflow for the emergency virtual visit to see the provider virtually. Patient demographics, consent, and other visit related information can be collected in the waiting room (e.g., as shown in FIG. 9 and FIG. 10).

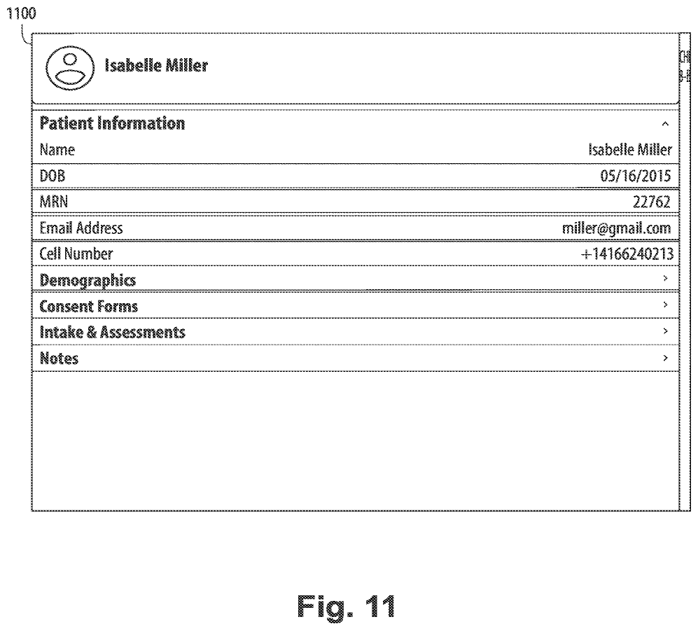

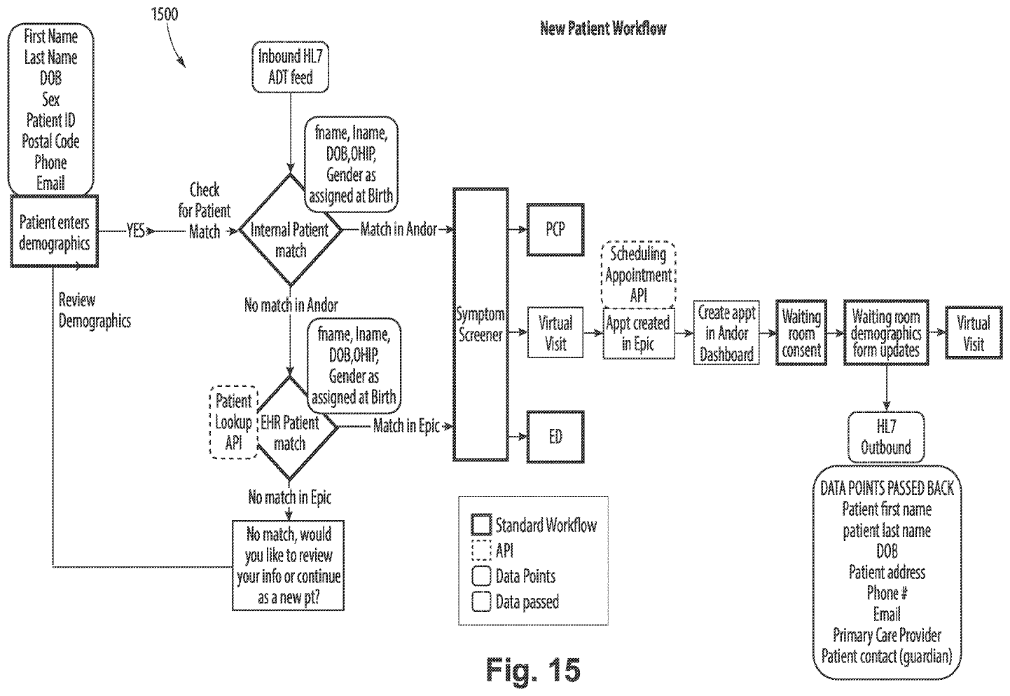

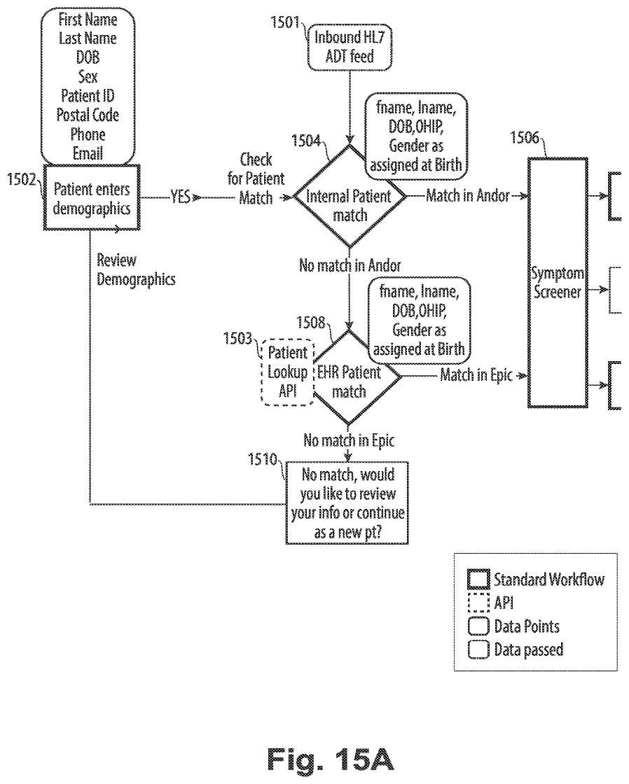

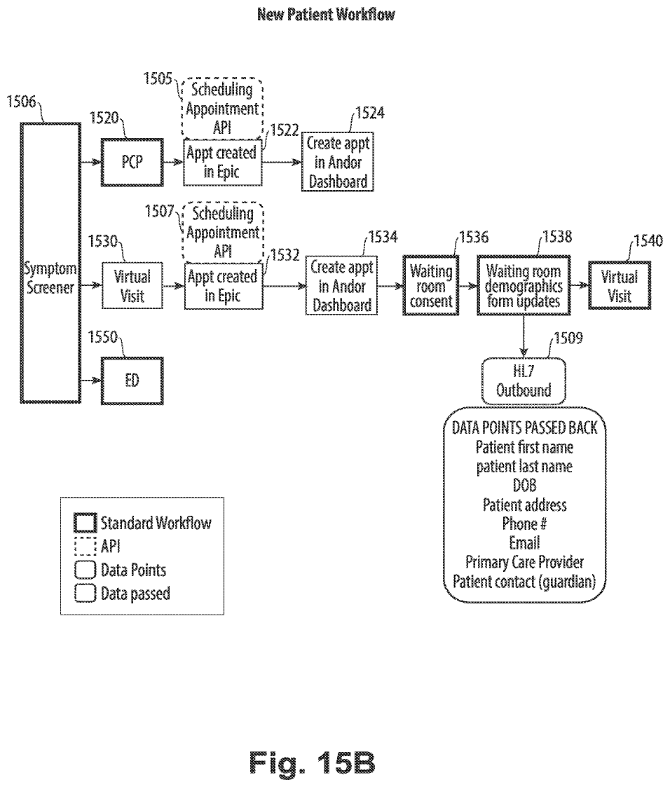

Staff or clinical users can see relevant information about the patient, such as the patient data and triage data. Examples are depicted in FIGS. 11, 12, and 13. An embodiment of a workflow to achieve this is explained in FIG. 15 (e.g., split and zoomed in FIGS. 15A and 15B).

### Embodiment of an Interface (e.g., a GUI)

Patient intake can begin with presentation of a demographics page for a user to enter and submit demographic information about a new or existing patient. The demographic information includes a patient's identifier that can be restricted, for example, to be sent via an Admission Discharge Transfer (ADT) feed that is interoperable with other patient care systems.

In certain embodiments, e.g., as shown in FIGS. 1A and 1B, there can be an age and/or zip code review at a suitable portion in the workflow, e.g., at the second step in the workflow. For example, patients outside a covered zip code area can may attempt to use the automated triage system, and this zip code can correlate to an emergency department (ED) list provided (e.g., described more below) in a “visit your emergency department” result.

New patients can be provided with a new patient page as shown in FIG. 2. For example, if the patient does not yet exist in the automated triage system, and opts to continue as a new patient, the new patient page provides an additional demographics page to fill out. Medical history information can be accessed using the patient's identification and/or entered by the user. Accordingly, patient data is obtained for the patient that includes the medical history information and the demographic information.

FIG. 3 shows a symptom screener GUI for answering triage questions (e.g., for information about primary and secondary medical complaints) can be presented to a user (e.g., the patient or a caretaker of the patient). The user can select primary and secondary medical complaints in the symptom screener GUI. An appointment service type (e.g., primary care service, specialist service, virtual emergency visit service, physical emergency room service) is recommended to the patent based on processing of the patient data and responses to the triage questions.

FIG. 4 shows an example emergency department (ED) result that is presented to the user. For example the user can receive an ED page with links to their nearest ED if an emergency condition is determined (e.g., whether they are a patient or not) based on the input symptoms.

FIG. 5 shows an example PCP result that is presented to the user. For example, in embodiments, the PCP result can include instruction to see a PCP if a primary care condition is determined (e.g., whether they are a patient or not) based on the input symptoms.

FIG. 6 shows an example emergency virtual visit page result that is presented only for eligible patients. For example, if the patient selects a primary and a secondary medical complaint that is determined to be a condition that can be treated by an emergency virtual visit, this can lead to a virtual visit page (e.g., during business hours), and the user can be asked to join the waiting room on their personal computing device.

FIG. 7 shows an example provider/staff view that can include the dashboard as shown in FIG. 7. A waiting room workflow can include a registration page, e.g., as shown in FIG. 8. The patient or the patient's caretaker can enter a waiting room and complete a registration process, for example.

The waiting room workflow can include an update patient demographics page, e.g., as shown in FIG. 9. An API can populate and present the patient's patient data at this point in the waiting room. The patient or the patient's caretaker can then be able to update and/or change the patient's demographics or medical history on this form. For example, PCP, PCP location, patient contacts (name, phone, address), can be entered as free text that is sent to be stored via HL7 standards.

The waiting room workflow can include an additional symptom related questions page, e.g., as shown in FIG. 10. For example, the patient or the patient's caretaker can continue on to answer screening questions related to the patient's current symptoms and other data related to the patient's medical complaint.

An example dashboard for the provider/staff view is shown in FIG. 11. Data about the patient can appear on the dashboard. The dashboard can provide patient statuses that allows clinicians to see where different patients are in the virtual waiting room. For example, REGISTRATION can signify that the associated patient is in the waiting room verifying demographics and completing forms. REGISTRATION COMPLETE can indicate that the patient has completed triage. JOIN can mean that triage is completed and the patient can now be seen by provider. IN PROGRESS can indicate that the patient and provider are in an in progress emergency virtual visit. COMPLETED can indicate that the emergency virtual visit has been successfully completed. ABANDONED can indicate that the emergency virtual visit was not joined after some set period of time (e.g., 1 hour).

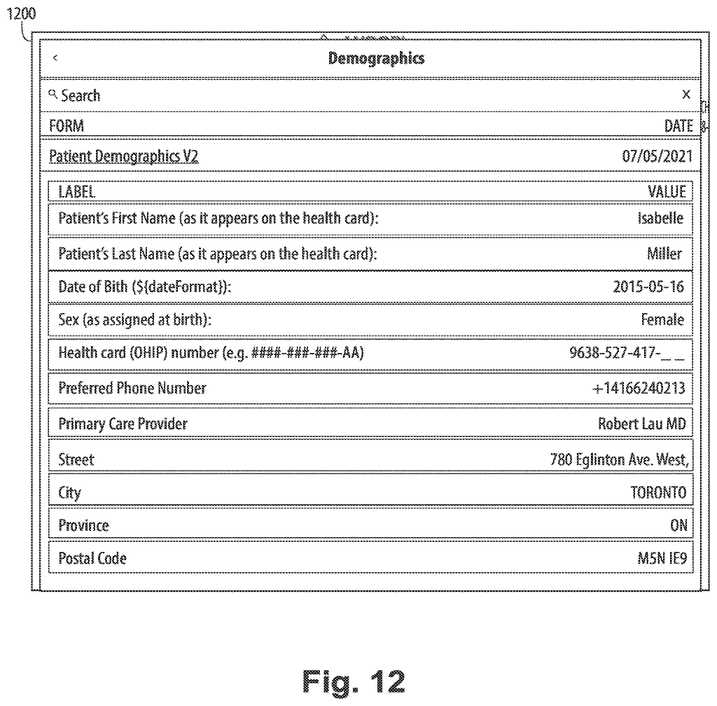

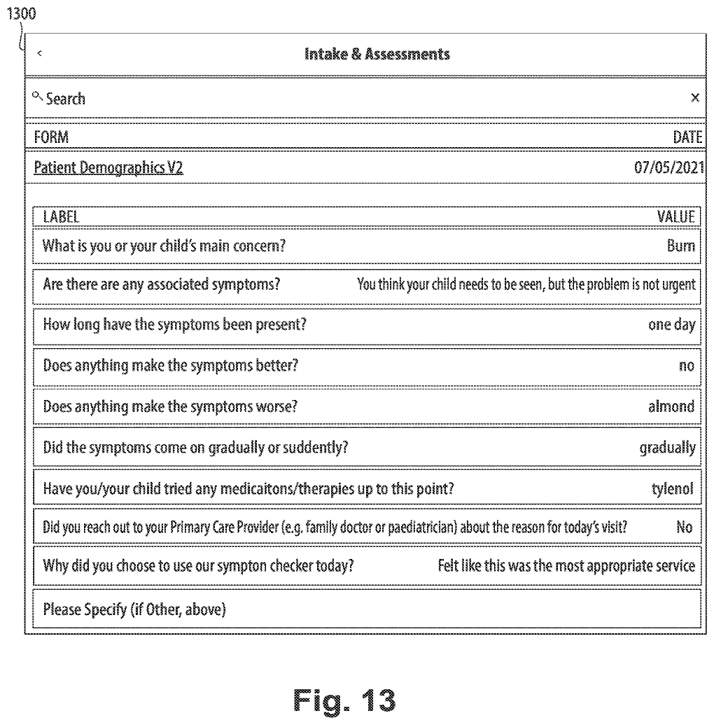

The provider/staff view can include a patient modal page, e.g., as shown in FIG. 12. In the patient modal page, that provider and staff can review patient information, demographics, consent forms, intake and assessments, and any notes, for example. The provider/staff view can include a patient modal intake page, e.g., as shown in FIG. 13.

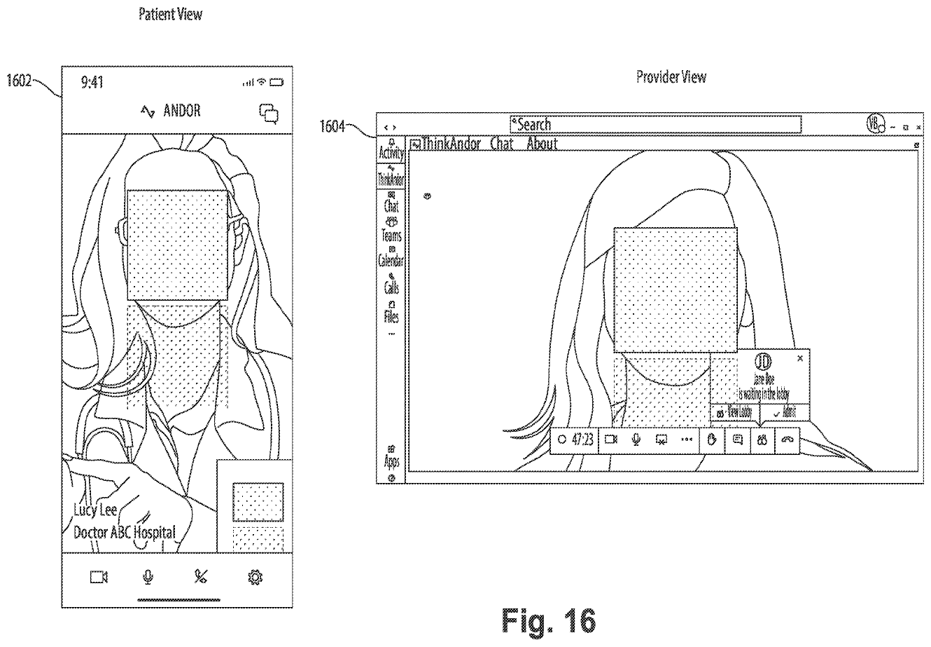

Once the patient has completed the registration process and the provider is ready to see the patient, the provider can click “Join” as shown in FIG. 7 and initiate the emergency virtual visit. This is depicted as a video call in FIG. 16.

The automated triage system can also set up non-emergency virtual visits for eligible patients with a PCP or specialist, including providing a non-emergency virtual visit page (not shown), a virtual waiting room or other means for providing or updating patient data and current patient symptoms at the user's leisure before the date for which the virtual visit is scheduled. The data entered can be transferred via API or other suitable means to the healthcare system at which the PCP or specialist practices, such as for providing a PCP or specialist dashboard with data about the patient. The information to be included in the PCP or specialist dashboard can include patient data and medical complaint information as well as status of progress preparing for and participating in the non-emergency virtual visit.

With reference now to FIGS. 15, 15A, and 15B, shown are flow diagrams demonstrating implementation of the various exemplary embodiments. It is noted that the order of operations shown in FIGS. 15, 15A, and 15B is not required, so in principle, the various operations may be performed out of the illustrated order. Also certain operations may be skipped, different operations may be added or substituted, some operations may be performed in parallel instead of strictly sequentially, or selected operations or groups of operations may be performed in a separate application following the embodiments described herein.

FIGS. 15, 15A, and 15B illustrate an embodiment of a workflow of the automated triage system. At block 1502, a method is performed to collect patient demographic information, such as name, date of birth, sex, patient ID, postal code, phone, and email. At blocks 1504 and 1506 an internal and external lookup procedure is performed, respectively, to see if patient information stored in an internal or external system include information about the patient. An inbound HL7 ADT feed can be used to perform the internal search. A patient lookup API can be used to perform the external search. If a match is found by the lookups performed at blocks 1504 or 1506, additional demographic information and medical history information can be accessed and populated into patient data that can be used for the patient, and the method continues at block 1506, If a match is not found by the lookups performed at blocks 1504 or 1506, at block 1510 a user (e.g., the patient or the patient's caretaker) can review and/or enter additional information to provide the patient data. The lookups performed at blocks 1504 or 1506 may be successful during subsequent attempts.

In some embodiments, the automated triage system is only available for patients that are registered with the system and that would have been found as an existing patient at blocks 1504 or 1506. If the patient was not found at blocks 1504 or 1506, the method can end. Alternatively, the method can continue at block 1506, albeit with reduced triaging, scheduling, and/or care services available to the patient.

At block 1506, key clinical data points such as the primary medical complaints and secondary medical complaints are collected as triage responses by a triaging process. The triage responses are processed using ML and/or by comparing the triage responses to data stored in a triage database to identify an entry that best matches the triage responses and to select the appointment service type associated with the identified entry. At one of blocks 1520, 1530, and 1550, the selected appointment service type is provided as a recommendation for care of the patient.

When the recommended appointment service type is PCP, the method continues at block 1520 in which the recommendation in which the recommendation for scheduling an appointment with a PCP (or alternatively a specialist) is provided to the user. At block 1522 an appointment can be created with a PCP, such as by using a scheduling appointment API at block 1505. At block 1524 appointment can be used to update the care team's dashboard.

When the recommended appointment service type is virtual visit, the method continues at block 1530 in which the recommendation for the virtual visit is provided to the user. At block 1532 an appointment can be created for the virtual visit, such as by using a scheduling appointment API at block 1507. The automated triage system can identify, based on the patient's input in insurance and/or payment fields of the demographic information, a payment method (using a particular insurance or through a one-time fee). The automated triage system through its API can automatically reconcile the insurance information and collect payment that can later be posted back into the health system's revenue cycle management system.

At block 1534, appointment can be used to update the care team's dashboard. At blocks 1536 and 1538, the patient can be assigned to the waiting room for the virtual visit, during which the patient can respond to prompts for consent for care and updates to demographics, medical symptoms, etc. Information input by a user at block 1538 can be used to update a clinical database, such as by using HL7 outbound. At block 1540 the virtual visit can be conducted. In the waiting room, the automated triage system allows the patient to invite additional participants, such as their family members, relatives or care givers to the virtual visit and when the virtual visit begins, the automated triage system can automatically invite the additional participant(s) and allow them to join the virtual visit by using a link provided to their phone or email. The link can be provided at the time or earlier by the automated triage system or separately by the patient. At block 1550, the method continues at block 1530 in which the recommendation is provided to the user for proceeding to the emergency room. The automated triage system can track and display via an ER dashboard a wait time of the patient. The ER dashboard can also display the triage responses and/or associated categories and sub-categories to help the physician to prioritize the patient. Furthermore, the automated triage system can dynamically prioritize the patient, making adjustments as additional patients are directed to the ER, based on patient data and/or triage responses of the patients. The automated triage system can compare the patients' wait times to the prioritizations and electronically allocate resources for the individual patients by control signals, such as by assigning ER rooms, ER equipment, and/or ER personnel to the individual patients accordingly. Accordingly, the automated triage system can send control signals to reserve and physically setup a particular ER room with particular physical equipment and/or digital charts that are designated for a particular patient. The automated triage system can additionally or alternatively send control signals to an electronic device used by medical practitioners assigned to work with a particular patient to direct them to the correct time and place, populate the electronic device with patient data and triage data about the patient, and/or additional information relevant to the particular patient's care.

In accordance with at least one aspect of this disclosure, the automated triage system can include at least one memory configured to store a plurality of programmable instructions and at least one processing device in communication with the memory, wherein the at least one processing device, upon execution of the plurality of programmable instructions is caused to automatically triage a patient based on received demographic data, a medical history of the patient, and a medical complaint about a present medical condition to triage the patient to determine a level of urgency of treatment needed by the patient and select an appointment service type for servicing the patient based on results of the triage to reduce unnecessary emergency room visits. The appointment service type can include a primary care physician type, a specialist type, an emergency virtual visit type, or an emergency room type, for example. A response with the appointment service type can be provided to the patient (e.g., via a GUI or via audio) and the appointment of the appointment service type selected for the patient can be initiated. Initiation of the appointment can depend on availability to the patient, such as whether the patient is eligible for the appointment and/or availability of the service type at an appropriate time and place.

The automated triage system can be configured to prompt the patient for, and to receive from the patient, key clinical data to identify the appointment service type to provide to the patient. The key clinical data can include a primary medical complaint and/or a secondary medical complaint, for example. The prompt for key clinical data can be based on at least one of the patient's demographic data and medical record.

The automated triage system can provide a GUI configured to be displayed on a patient device display to interact with a user, including receiving user input (e.g., via a touchscreen of a console or a user device). In certain embodiments, the automated triage system can be configured to automatically collect patient related information from an electronic medical record database.

In certain embodiments, the automated triage system can be configured to determine if and validate that the patient is an existing patient or determine if the patient is a new patient. This determination is used to provide either an existing patient GUI pane or a new patient GUI pane. The patient can be prompted based on whether the patient is an existing patient or a new patient. The automated triage system can be configured to provide a full record of care to the patient or a provider of a patient upon determining that a patient is an existing patient.

In certain embodiments, the automated triage system can be configured to compare the user inputs to a triage database. The selection of the appointment service type can be a function of a result of the comparison. In certain embodiments, the automated triage system can apply ML and/or AI to select the appointment service type.

In certain embodiments, the at least one processing device can be configured to automatically recommend a service provider from a plurality of candidate service providers as a function of one or more of patient affiliation with a service provider of the plurality of candidate service providers; acceptance of patient insurance, location of the service provider; availability of one or more of a specialized practitioner, facilities, and/or equipment; cost quote or estimate; type of payment accepted; and patient preference.

In certain embodiments, initiating the appointment can include outputting at least one control signal to order, allocate, or reserve at least one resource for performance of the appointment.

In certain embodiments, initiating the appointment can include prioritizing patients based on results of the triage and tracking wait time of the patient at the appointment as a function of prioritization of the patient.

In certain embodiments, initiating the appointment can include prioritizing patients based on results of the triage and outputting at least one control signal to order, allocate, or reserve at least one resource for performance of the appointment as function of the prioritization of the patients.

In certain embodiments, the appointment is a virtual visit and initiating the appointment includes automatically providing a link to patient selected invitees to join the virtual visit.

In accordance with at least one further aspect of the disclosure a method is provided. The method includes displaying a patient identification GUI having one or more fields configured to receive identification and/or location information from a patient, displaying a triage GUI having one or more fields configured to receive medical complaint information, determining a virtual care condition existence based on the medical complaint information, displaying a virtual visit screen where a virtual care condition exists, and displaying an alternate screen where no virtual care condition exists.

In certain embodiments, the alternate screen can include an emergency department screen configured to provide instruction to the user to go to an emergency department and/or emergency department information based on location information if an emergency condition exists. The alternate screen can include a primary care physician screen instructing a user to contact their primary care physician if no emergency condition or virtual care condition exists.

In another aspect of the disclosure a non-transitory computer readable medium storing executable code that is configured to cause the at least one processing device to perform the disclosed method.

A system can include one or more computer modules configured to display a patient identification graphical user interface (GUI) having one or more fields configured to receive identification and/or location information from a patient, display a triage GUI having one or more fields configured to receive medical complaint information, determine a condition type based on the medical complaint information, wherein the condition type is one of a virtual care condition, an emergency condition, or a primary care condition, display a virtual visit screen if a virtual care condition exists, display an emergency department screen if an emergency condition exists, and display a primary care physician screen if a primary care condition exists. In certain embodiments, the virtual care screen can include a join waiting room button configured to place the user into a virtual waiting room for a virtual care visit.

Any suitable module can include any suitable hardware and/or software module(s) configured to perform the associated function and/or any other suitable function(s). Any suitable method disclosed herein can be implemented on a non-transitory computer readable medium. Any suitable interfaces (e.g., GUI's) are contemplated herein.

As will be appreciated by those skilled in the art, aspects of the present disclosure may be embodied as a system, method or computer program product. Accordingly, aspects of this disclosure may take the form of an entirely hardware embodiment, an entirely software embodiment (including firmware, resident software, micro-code, etc.), or an embodiment combining software and hardware aspects, all possibilities of which can be referred to herein as a “circuit,” “module,” or “system.” A “circuit,” “module,” or “system” can include one or more portions of one or more separate physical hardware and/or software components that can together perform the disclosed function of the “circuit,” “module,” or “system”, or a “circuit,” “module,” or “system” can be a single self-contained unit (e.g., of hardware and/or software). Furthermore, aspects of this disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Computer program code for carrying out operations for aspects of this disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of this disclosure may be described above with reference to flowchart illustrations and/or block diagrams of methods, apparatus (systems) and computer program products according to embodiments of this disclosure. It will be understood that each block of any flowchart illustrations and/or block diagrams, and combinations of blocks in any flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in any flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

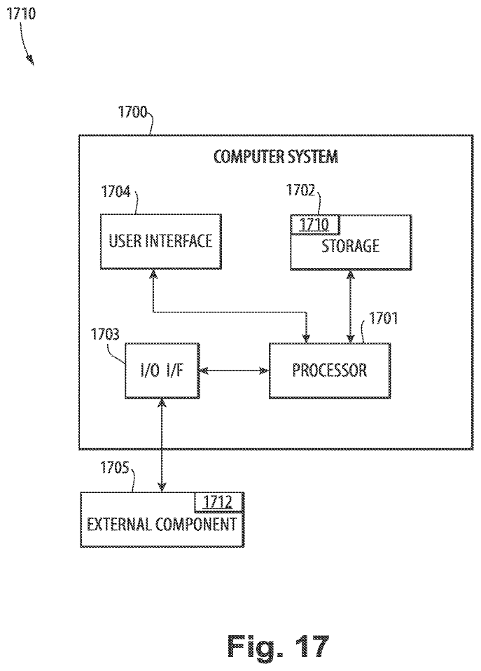

With reference to FIG. 17, a block diagram of the automated triage system is shown as example automated triage system 1710 with an example computer system 1700. Computer system 1700 can be embodied separately or in a combination of one or more computer systems. In various embodiments, computer system 1700 may be a server, a mainframe computer system, a workstation, a network computer, a desktop computer, a laptop, a handheld computer, an embedded system, or the like, and/or include one or more of a processor, field-programmable gate array (FPGA), application specific integrated circuit (ASIC), microcontroller, microprocessor, or the like. Computer system 1700 is only one example of a suitable system and is not intended to suggest any limitation as to the scope of use or functionality of embodiments of the disclosure described herein. Computer system 1700 can be implemented using hardware, software, and/or firmware. Regardless, computer system 1700 is capable of being implemented and/or performing functionality as set forth in the disclosure.

Computer system 1700 is shown in the form of a general-purpose computing device. Computer system 1700 includes one or more processors 1701, storage 1702, an input/output (I/O) interface (I/F) 1703 that can communicate (directly or via processor 1701) with an internal component, such as a user interface 1704, and an external component 1705.

Automated triage system 1710 can be configured to handle large amounts of data. Hence, computer system(s) 1700 can be implemented, for example, using multiprocessors, a big data architecture, or one or more cloud-based computer systems.

Processor(s) 1701 can include, for example, a single core or multicore processor, a programmable logic device (PLD), microprocessor, DSP, a microcontroller, an FPGA, an ASIC, and/or other discrete or integrated logic circuitry having similar processing capabilities. Processor(s) 1701 and storage 1702 can be included in components provided in the FPGA, ASIC, microcontroller, or microprocessor, for example. Storage 1702 can include, for example, volatile and non-volatile memory for storing data temporarily or long term, and for storing programmable instructions 1710 executable by the processor(s) 1701 for causing performance of the disclosed methods. Storage 1702 can be a removable (e.g., portable) memory for storage of program instructions. I/O I/F 1703 can include an interface and/or conductors to couple to the one or more internal components and/or external components 1705. External components 1705 can include one or more triage databases 1712, for example.

The program instructions include program modules for generally carrying out the functions and/or methodologies of embodiments of the disclosure as described herein, as well as an operating system, one or more application programs, other program modules, and program data. Each of the operating system, one or more application programs, other program modules, and program data or some combination thereof, may include an implementation of a networking environment.

The computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flow diagram and/or block diagram block or blocks.

The computer program instructions may also be loaded onto a computer, other programmable data processing apparatus, or other devices to cause a series of operational operations to be performed on the computer, other programmable apparatus or other devices to produce a computer implemented process. The instructions when executed on the computer or other programmable apparatus provide processes for implementing the disclosed functions/acts, including those specified in the block diagram block or blocks.

Computer system 1700 is only one example of a suitable system and is not intended to suggest any limitation as to the scope of use or functionality of embodiments of the disclosure described herein. Regardless, computer system 1700 is capable of being implemented and/or performing any of the functionality set forth hereinabove.

Computer system 1700 may be described in the general context of computer system-executable instructions, such as program modules, being executed by a computer system. Generally, program modules may include routines, programs, objects, components, logic, data structures, and so on that perform particular tasks or implement particular abstract data types. **

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. An automated triage system comprising:
at least one memory configured to store a plurality of programmable instructions; and
at least one processing device in communication with the at least one memory; and
wherein the at least one processing device, upon execution of the plurality of programmable instructions is caused to automatically:
receive demographics data for a patient,
access or receive a medical history for the patient,
receive a medical complaint about a present medical condition,
triage the patient based on the demographic data, the medical history, and the medical complaint to determine a level of urgency of treatment needed by the patient,
select an appointment service type for servicing the patient based on results of the triage to reduce unnecessary emergency room visits,
provide a response to the patient including the appointment service type selected, and
initiate an appointment of the appointment service type selected for the patient that is available to the patient.

2. The system of claim 1 wherein the appointment service type includes a primary care physician type, a specialist type, an emergency virtual visit type, or an emergency room type.

3. The system of claim 1 wherein the at least one processing device is further caused to prompt the patient, based on at least one of the demographic data and the medical record, for key clinical data, wherein receiving the medical complaint includes receiving the key clinical data.

4. The system of claim 3 wherein the key clinical data includes a primary medical complaint and a secondary medical complaint.

5. The system of claim 1 wherein the at least one processing device is further configured to generate a graphical user interface configured to be displayed on a display device for interacting with a user and receiving user input.

6. The system of claim 3 wherein the at least one processing device is further caused to determine if the patient is an existing patient or a new patient, wherein prompting the patient is further based on the determination.

7. The system of claim 6, wherein the at least one processing device is further caused to provide a full record of care for analysis to the patient or to a provider of the patient, upon determining that the patient is an existing patient.

8. The system of claim 1 wherein the at least one processing device is further caused to compare the user inputs to a triage database, wherein selection of the appointment service type is a function of a result of the comparison.

9. The system of claim 1 wherein the at least one processing device is configured to apply machine learning to select the appointment service type.

10. The system of claim 1 wherein the at least one processing device is configured to automatically recommend a service provider from a plurality of candidate service providers as a function of one or more of: patient affiliation with a service provider of the plurality of candidate service providers; acceptance of patient insurance; location of the service provider; availability of one or more of a specialized practitioner, facilities, or equipment; cost quote or estimate; type of payment accepted; or patient preference.

11. The system of claim 1 wherein initiating the appointment includes outputting at least one control signal to order, allocate, or reserve at least one resource for performance of the appointment.

12. The system of claim 1 wherein initiating the appointment includes prioritizing patients based on results of the triage and tracking wait time of the patient at the appointment as a function of prioritization of the patient.

13. The system of claim 1 wherein initiating the appointment includes prioritizing patients based on results of the triage and outputting at least one control signal to order, allocate, or reserve at least one resource for performance of the appointment as function of the prioritization of the patients.

14. The system of claim 1 wherein the appointment is a virtual visit and initiating the appointment includes automatically providing a link to patient selected invitees to join the virtual visit.

15. A method comprising:
displaying a patient identification graphical user interface having one or more fields configured to receive identification and location information from a patient;
displaying a triage graphical user interface having one or more fields configured to receive medical complaint information;
determining a virtual care condition existence based on the medical complaint information;
displaying a virtual visit screen where a virtual care condition exists; and
displaying an alternate screen where no virtual care condition exists.

16. The method of claim 15 wherein the alternate screen includes an emergency department screen configured to provide instruction to the user to go to an emergency department and emergency department information based on location information if an emergency condition exists.

17. The method of claim 16 wherein the alternate screen includes a primary care physician screen instructing a user to contact their primary care physician if no emergency condition or virtual care condition exists.

18. The method of claim 15 further comprising a non-transitory computer-readable medium storing executable code configured to cause at least one processor to perform the method.

19. A system comprising one or more computer modules configured to:
display a patient identification graphical user interface having one or more fields configured to receive identification or location information from a patient;
display a triage graphical user interface having one or more fields configured to receive medical complaint information;
determine a condition type based on the medical complaint information, wherein the condition type is one of a virtual care condition, an emergency condition, or a primary care condition;
display a virtual visit screen if a virtual care condition exists;
display an emergency department screen if an emergency condition exists; and
display a primary care physician screen if a primary care condition exists.

20. The system of claim 19 wherein the virtual care screen includes a join waiting room button configured to place the user into a virtual waiting room for a virtual care visit.
